(19) United States
(12) Patent Application Publication
(10) Pub. No.: US 2006/0046998 A1
(43) Pub. Date: Mar. 2, 2006

(54) Method of treatment of gastroesophageal reflux disease and laryngopharyngeal reflux disease

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials, LLC, Bridgeport, CT (US)

(21) Appl. No.: 11/210,865

(22) Filed: Aug. 25, 2005

Related U.S. Application Data
(60) Provisional application No. 60/604,006, filed on Aug. 25, 2004.
(60) Provisional application No. 60/628,547, filed on Nov. 18, 2004.

(51) Int. Cl. A61K 31/4439

(52) U.S. Cl. 514/338

(57) ABSTRACT

The present invention relates to compositions and methods for initial treatment of GERD and LPRD using a proton pump inhibitor drug administered concomitantly with a histamine H₂ receptor inhibitor, either transdermally or orally.

This application claims the benefit of prior co-pending U.S. Provisional Application Ser. No. 60/604,006, filed Aug. 25, 2004 and prior co-pending U.S. Provisional Application Ser. NO. 60/628,547, filed Nov. 18, 2004.

### BACKGROUND OF THE INVENTION

1. Technical Field

This invention relates to the field of medical treatment, and specifically to a method for treatment of gastroesophageal reflux disease (GERD) and laryngopharyngeal reflux disease (LPRD) which is particularly useful for initial treatment of these conditions.

2. Description of the Background Art

Gastroesophageal reflux is a normal phenomenon that becomes pathologic when severe or frequent enough to result in pain or damage to the esophagus. Laryngopharyngeal reflux refers to gastroesophageal reflux that moves up the esophagus through its upper sphincter and into the back of the throat. The most common symptom of GERD is heartburn, sometimes accompanied by noticeable regurgitation of the stomach contents. Damage to the esophagus that can result from gastroesophageal reflux as part of GERD and/or LPRD includes esophageal erosion, esophageal ulcer, esophageal stricture, and replacement of normal esophageal epithelium with abnormal (Barrett's) epithelium, which may lead to esophageal cancer. Aspiration of acidic material in GERD or in LPRD also can occur, and refluxate may be present in and cause damage or irritation to, for example, the oropharynx, nasopharynx, sinuses, larynx, teeth and gums. Additional symptoms may include pain on swallowing, coughing, asthma, gingivitis, hoarseness or earache. Because these acid reflux syndromes are fairly common and result in considerable morbidity, improvements in treatments for them is desirable in the art.

Oral proton pump inhibitors (PPI), which inhibit the H⁺-K⁺ ATPase, have been used previously to treat GERD and LPRD. These drugs are considered the drugs of choice for treatment of both GERD and LPRD, but have unfortunate drawbacks. Persons taking oral PPI drugs often must wait many hours and usually days (for example, four days for esomeprazole (Nexium™) and five days for lansoprazole (Prevacid™)) to obtain maximal symptomatic relief when beginning a course of PPI therapy. Therefore, occasional use of PPI drugs often may not provide any meaningful symptomatic relief. PPI drugs must be taken for several days (up to two weeks or more is recommended) each time symptoms occur, or must be taken at a very high dose, such that unpleasant side effects are more likely to occur, at least in the short term. Persons taking oral PPIs, which have very short half-lives, usually are subject to fluctuations in drug plasma concentration for an undesirably long period of time. This phenomenon can diminish the drugs, effectiveness. Therefore, there is a need in the art for a method to overcome the disadvantages of traditional PPI therapy for GERD and LPRD.

Histamine H₂ receptor antagonists (H₂RA) also are per se known in the art for treatment of symptoms of and syndromes related to elevated gastric acid, for example in gastric ulcer, GERD, LPRD and the like. H₂RA drugs usually achieve therapeutic blood levels within one hour after oral dosing and provide symptomatic relief within 2-11 hours after dosing, with an average of 4 hours. The H₂RA drugs can provide symptomatic relief more rapidly than PPI drugs, however they are considered inferior to PPIs in terms of both healing of erosions in the gastrointestinal tract and symptom relief in general. Therefore, H₂RA drugs are used primarily to treat non-erosive or only mildly erosive GERD or LPRD and are not considered first-line therapy. In addition, their side effects make them generally unsuitable for long-term treatment.

Prokinetic (promotility) medications such as cisapride, metoclopramide and urecholine also are used occasionally in the treatment of GERD and LPRD. These drugs increase the pressure of the lower esophageal sphincter, reducing reflux of stomach contents into the esophagus and therefore act in theory to prevent the root cause of esophageal damage seen in GERD and LPRD. These medications also, however, are not considered first-line therapy, due to some lack of effectiveness and unacceptable side effects. Cisapride has been removed from the market for unacceptable side effects.

Traditional antacid therapy also is known for symptomatic relief of hyperacid conditions such as GERD and LPRD. Over-the-counter antacids are commonly used, and are effective for short-term relief of painful symptoms in most cases, but do not provide the healing benefits of either PPIs or H₂RAs.

Oral administration of the drugs discussed above is known. Transdermal administration of drugs generally also is known in the art. This route of pharmaceutical administration has several advantages, including avoiding first pass metabolism. Other advantages of transdermal administration include the ability to consistently administer the drug at a zero-order (or at least nearly zero-order) rate and the ability to remove the drug rapidly from the user in cases of adverse effects, toxicity, overdose or any other undesirable effect. An additional advantage to transdermal administration is increased patient compliance, since administration is required less frequently. At the present time, a transdermal dosage form may be designed to administer drugs for up to seven consecutive days, eliminating the need for the inconvenience of daily or twice daily administrations which are needed when administering oral forms of drugs with short half-lives, such as PPIs and H₂RAs. Furthermore, fluctuations in plasma levels that can occur with oral dosing may be greatly diminished with transdermal administration of a drug. Peaks and troughs in plasma concentration on oral delivery are more pronounced with drugs which have fairly short half- lives, and therefore can be problematic in traditional therapy for GERD and LPRD.

Particular types of transdermal drug delivery devices have been described previously. In drug-in-adhesive patches, a drug is dissolved or suspended directly in the adhesive which contacts the skin. Reservoir transdermal systems include a liquid or semi-liquid compartment containing a drug suspension or solution, separated from the skin by a semi-permeable membrane. In matrix transdermal systems, a drug is contained within a solid or semi-solid matrix which contacts the skin of the user and is surrounded at the perimeter by an adhesive. These different transdermal systems and methods for their manufacture and testing are described in, for example, U.S. Pat. Nos. 4,751,087; 5,405,317; 6,312,715; and 6,322,532, the disclosures of which are hereby incorporated by reference. Transdermal devices for use with the invention may contain skin penetrant compounds, softeners, solvents and other excipients and an occlusive or non-occlusive backing.

Current therapy for GERD and LPRD (monotherapy with a PPI) is generally effective in most patients, however symptomatic relief often is undesirably delayed, resulting in slower healing and unnecessary pain to sufferers of GERD and LPRD. Therefore, there is a need in the art for a method of treatment for GERD and LPRD, and particularly for initial treatment of GERD and LPRD, which overcomes the disadvantages of currently available therapy. It would be desirable to provide a drug therapy for GERD and LPRD which provides rapid symptom relief while still achieving sufficient healing of any esophageal erosions, particularly in the initial phase of treatment. It further would be desirable to provide an additional therapeutic benefit by designing a regimen that can reduce delayed healing and the appearance of side effects caused by prolonged periods of drug concentration peaks and troughs and which provides a convenient form for delivery of this therapeutic regimen.

### SUMMARY OF THE INVENTION

Accordingly, in one preferred embodiment, this invention is directed to a method for initial treatment of gastroesophageal reflux disease or laryngopharyngeal reflux in a mammal which comprises administering concomitantly a proton pump inhibitor drug and a histamine H₂ receptor antagonist drug for about 5 days to about 15 days, about 7 days to about 14 days, or about one week or two weeks, and thereafter continuing to administer a proton pump inhibitor drug, indefinitely, for 1-12 weeks, for 1-3 weeks or for about one week or three weeks. In preferred embodiments, the total duration of said initial treatment of gastroesophageal reflux disease or laryngopharyngeal reflux disease is about 2 to about 12 weeks.

The methods may be practiced with proton pump inhibitor drugs and/or histamine H₂ receptor antagonist drugs each formulated for oral administration or for transdermal administration, using separate formulations for each drug or wherein the proton pump inhibitor drug and the histamine H₂ receptor antagonist drug are formulated in single unit dose formulations (“fixed combinations”), that is wherein a single unit dose formulation contains both the proton pump inhibitor drug and the histamine H₂ receptor antagonist drug, either for oral or transdermal administration.

Preferred methods are practiced wherein the proton pump inhibitor drug is selected from the group consisting of omeprazole, esomeprazole, lansoprazole, raveprazole and pantoprazole and the histamine H₂ receptor antagonist drug is selected from the group consisting of ranitidine, famotidine, nizatidine and cimetidine.

In yet another embodiment, this invention is directed to a dosage form for initial treatment of gastroesophageal reflux disease or laryngopharyngeal reflux disease in a mammal which comprises a proton pump inhibitor drug; a histamine H₂ receptor antagonist drug; and a pharmaceutically acceptable carrier. The proton pump inhibitor drug may be selected from the group consisting of omeprazole, esomeprazole, lansoprazole, raveprazole and pantoprazole and the histamine H₂ receptor antagonist drug may be selected from the group consisting of ranitidine, famotidine, nizatidine and cimetidine. These dosage forms may be oral dosage forms or transdermal dosage forms.

In yet a further embodiment, this invention provides a method of treating initial gastroesophageal reflux disease or laryngopharyngeal reflux disease in a mammal which comprises administering to the mammal a dosage form as described immediately above.

In yet a further embodiment, this invention is directed to a kit for initial treatment of gastroesophageal reflux disease or laryngopharyngeal reflux disease in a mammal which comprises a dosage form as described above and a proton pump inhibitor drug. Kits of preferred embodiments may comprise a one-week supply of a histamine H₂ receptor antagonist drug and a two-week supply of a proton pump inhibitor drug or a one-week supply of a histamine H₂ receptor antagonist drug and a four-week supply of a proton pump inhibitor drug.

In yet a further embodiment, this invention is directed to a kit for initial treatment of gastroesophageal reflux disease or laryngopharyngeal reflux disease which comprises a 5- to 15-day supply of a histamine H₂ receptor antagonist drug and a 12- to 99-day supply of a proton pump inhibitor drug. Preferred embodiments of this aspect of the invention contain a 1- to 2-week supply of a histamine H₂ receptor antagonist drug and a 2- to 12-week supply of a proton pump inhibitor drug or more preferably a 1 week supply of a histamine H₂ receptor antagonist drug and a 2 week supply of a proton pump inhibitor drug or a 1 week supply of a histamine H₂ receptor antagonist drug and a 4-week supply of a proton pump inhibitor drug. Preferred proton pump inhibitor drugs are selected from the group consisting of omeprazole, esomeprazole, lansoprazole, raveprazole and pantoprazole and preferred histamine H₂ receptor antagonist drugs are selected from the group consisting of ranitidine, famotidine, nizatidine and cimetidine.

In yet a further embodiment, this invention is directed to a kit for initial treatment of gastroesophageal reflux disease or laryngopharyngeal reflux disease which comprises a 5- to 15- day supply of one or more combination dosage forms each comprising a histamine H₂ receptor antagonist drug and a proton pump inhibitor drug and (b) 1-to 12-week supply of a proton pump inhibitor drug. The combination dosage form may be a trandermal dosage form or an oral dosage form.

### DETAILED DESCRIPTION OF THE PREFERRED EMBODIMENTS

This invention provides, in some embodiments, pharmaceutical combination kits and oral or transdermal drug dosage forms which contain a proton pump inhibitor drug and a histamine H₂ receptor antagonist drug. These two drugs may be contained in the same oral or transdermal dosage form or in two separate dosage forms. The invention also relates to a method for therapy of GERD and LPRD, which is particularly useful as a first-line or initial therapy, comprising administering a combination therapeutic regimen as described in the kits and dosage forms discussed here. “First-line” or “initial” treatment refers to treatment in the first instance after a new diagnosis of GERD or LPRD, or after a relapse of GERD or LPRD following cessation of treatment, however the treatment method can be useful in any GERD or LPRD patient who is not responding to monotherapy with PPIs or H₂RAs.

PPI drugs are substituted benzimidazole compounds that specifically inhibit gastric acid secretion by affecting the H⁺/K⁺ ATPase enzyme system (the proton pump). These drugs, for example esomeprazole, are rapidly absorbed and have very short half-lives, however they exhibit prolonged binding to the H⁺/K⁺ ATPase enzyme. The anti-secretory effect reaches a maximum in about 4 days with once-daily dosing. Because of these characteristics, patients beginning PPI therapy do not receive maximum benefit of the drug and healing may not begin for up to 5 days after therapy begins when PPIs are used alone for initial therapy of GERD or LPRD.

The phrase “proton pump inhibitor” (PPI) refers to any compound that inhibits the H⁺-K⁺ ATPase of gastric parietal cells. PPIs known at the present time include, for example, omeprazole, esomeprazole, lansoprazole, raveprazole, pantoprazole, or optically pure isomers thereof. Active metabolites and pro-drugs of these compounds also are included in the phrase. Active metabolites include, but are not limited to, hydroxy-omeprazole, hydroxylansoprazole, omeprazole carboxylic acid, desmethyl pantoprazole and optically pure isomers thereof. Exemplary PPI compounds and methods for their synthesis are available in the prior art, for example, in U.S. Pat. Nos. 4,544,750; 4,758,579; 5,045,552; 5,374,730 and 5,386,032, the disclosures of which are hereby incorporated by reference.

H₂RA drugs, such as ranitidine and cimetidine, generally are competitive, reversible inhibitors of histamine at histamine H₂ receptors in gastric cells and at other locations in the body. These compounds inhibit basal gastric acid secretion and gastric acid secretion stimulated by food and other stimuli. H₂RA drugs have been used for maintenance therapy in GERD, LPRD or in gastric or duodenal ulcers, however tolerance (attenuated antisecretory activity) has been observed to develop with long-term use, or even use of only 2 weeks. See, for example, Komazawa et al., J. Gastroenterol. Hepatol. 18(6):678-682, 2003. In addition, liver enzyme elevations and occasional pancreatitis side effects have been of some concern during long-term use. Therefore, use of H₂RA drugs for long-term therapy of GERD and LPRD generally is not recommended.

The phrase “H₂ receptor antagonist” or “histamine H₂ receptor antagonist, (H₂RA) refers to any compound that inhibits the interaction or efficacy of histamine with H₂ receptors. H₂RA drugs include, for example, cimetidine, ranitidine, famotidine, nizatidine and optically pure isomers thereof, which are known per se in the art. Also included in the definition of this phrase are active metabolites and pro-drugs of known H₂RA compounds, for example, N2-monodesmethylnizatidine. Some non-limiting exemplary compounds and methods for their preparation are described in, for example, U.S. Pat. Nos. 5,700,945; 5,118,813; 4,413,129; 4,855,439; 4,886,910 and 4,886,912, the disclosures of which are hereby incorporated by reference.

The addition of an H₂RA drug to a regimen for initial treatment with a PPI provides more rapid decrease in the patient's synthesis of stomach acid, providing relief of symptoms in the short term prior to the time when the PPI drug takes full effect. This decreases the uncomfortable waiting period of a newly diagnosed GERD or LPRD patient, a relapsed GERD or LPRD patient or any patient suffering from the painful symptoms of acid reflux and speeds healing of any damage caused by gastric acid. The two drugs of the invention are administered concomitantly according to the invention. The term “concomitantly” includes simultaneous or sequential administration; the two drugs need not be administered at the same time, but preferably the two drugs are administered within a 24 hour period or at close enough proximity in time such that measurable serum concentrations of each drug are present for at least a portion of that 24 hour period. For example, when oral dosage forms designed for once-daily administration are administered, each drug may be administered at any time of day. Preferably, the PPI drug is administered in the morning and the H₂RA drug is administered in the evening. Likewise, transdermal drug delivery devices may be applied to the skin at the same time or at different times, so long as the devices are able to provide, within about 4 days and preferably within about 1 day or less, a serum concentration of each drug which is effective to provide symptomatic relief of GERD and LPRD.

The invention is particularly useful in initial treatment of GERD or LPRD or treatment of relapse of GERD or LPRD symptoms. Transdermal administration of a PPI drug decreases the likelihood of potential side effects and provides a more rapid, effective steady-state plasma concentration of drug in a patient's body. Embodiments in which the drugs are formulated as transdermal drug delivery devices, for transdermal administration, therefore form part of this invention.

The phrase “transdermal drug delivery device” refers to any dosage form suitable for transdermal administration of a pharmaceutical compound. Included in this phrase are any of the known types of transdermal dosage forms, including but not limited to drug-in-adhesive, matrix, reservoir or other transdermal devices, commonly known as patches, or any other preparation designed for transdermal delivery of an active pharmaceutical compound. As used in this application, the term “patch” is intended to be interchangeable with the phrase “transdermal drug delivery device” and encompasses all dosage forms for transdermal delivery of a drug.

Transdermal drug delivery devices for use with the invention can be of any design known in the art, including specialized patches for iontophoretic delivery or in conjunction with small electric currents (electroporation), ultrasound or microneedle technology to assist delivery across the skin. Suitable patches may include any type of transdermal device technology known to the art, with or without a rate-limiting membrane to control diffusion of the active ingredient(s) to the skin. Transdermal drug delivery devices for delivery of more than one drug from a single dosage form can be constructed with a single reservoir, matrix or adhesive which contains both drugs, or if biostability or other compatibility problems exist, can be constructed with two separate reservoirs or matrices, one for each compound. Exemplary suitable transdermal technologies which are compatible with the present invention include those used in, for example, D-TRANS™, E-TRANS™, MICROFLUX™, LATITUDE™, LATITUDE™ DUO, CLIMARA PRO™, for example. Any known type of transdermal delivery device or system may be used with the embodiments of this invention.

Oral dosage forms are well known in the art and have been used to administer these drugs. Any of the known oral dosage forms of proton pump inhibitor drugs and histamine H₂ receptor inhibitor drugs are suitable for use with this invention, however the skilled person can formulate mono- or dual-therapy oral dosage forms according to the invention using known pharmaceutical excipients.

In the combination therapies of this invention, suitable. plasma concentrations of PPI drug generally are in the range used in single therapy with oral administration as in the prior art. Lower doses also may be used since in combination therapy the two drug compositions have additive and/or synergistic effects. As well, higher doses of PPI may be used with certain transdermally administered embodiments of this invention since the undesirable peaks in plasma drug concentration seen when a PPI is administered orally are diminished when it is administered transdermally.

Dosages and desirable steady-state plasma concentrations of both PPIs and H₂RAs can be determined by any skilled physician or other person of skill in the art. These drugs are per se known, therefore the art has some experience in determining effective and preferred dosages. There is experience in the prior art in formulating oral dosage forms of PPIs and of H₂RAs. Any of these known dosage forms are suitable for use with the invention. Persons of skill in the art are able to formulate fixed combination drug formulations for oral delivery of a PPI and an H₂RA. A “fixed combination” is a single pharmaceutical unit for administration that contains more than one active ingredient, where each active ingredient is present in a predetermined or “fixed” amount. Such a fixed combination may be formulated for any route of administration.

Combinations of excipients as are known in the art may be used for direct compression, wet or dry granulation or any other oral dosage forms, including tablets, capsules, caplets, lozenges, and the like, as well as liquid dosage forms for oral delivery. Suitable classes of excipients include, for example, fillers and diluents, compression aids, disintegrants, lubricants, glidants, binders, buffers, flavorants, dyes and colorants, perfumes and the like.

Generally, PPI drugs are administered orally daily in doses of about 5 mg to about 150 mg, preferably from about 10 mg to about 80 mg and most preferably about 10 mg to about 40 mg. The doses may be divided. Desirable plasma concentrations generally are in the range of about 1.0 μmol/L to about 10.0 μmol/L. Preferred steady-state plasma concentrations of PPI drug are about 2.0 μmol/L to about 8.0 μmol/L or about 3.0 μmol/L to about 5.0 μmol/L. H₂RA drugs generally are administered orally in amounts of about 25 mg to about 500 mg twice daily, preferably in amounts of about 50 to about 300 mg twice daily and most preferably in amounts of about 75 mg to about 250 mg twice daily. For H₂RA drugs, serum concentrations of about 36 ng/mL to about 94 ng/mL are known to inhibit 50% of stimulated gastric acid secretion. Desirable steady-state plasma concentrations of H₂RA drugs for use with embodiments of this invention are about 30 ng/mL to about 750 ng/mL, preferably about 50 ng/mL to about 500 ng/mL and most preferably about 100 ng/mL to about 400 ng/mL. An “effective amount” of these drugs therefore is determined using this information as a guide. Generally these ranges of doses and serum or plasma concentrations are effective amounts of the compositions, however the term “effective amount” also encompasses any amount which is sufficient to produce amelioration of symptoms and/or healing of erosions if present.

Treatment with the PPI/H₂RA combination treatment products, kits and devices of this invention may continue for one week, two weeks, three weeks, four weeks, eight weeks or sufficient time to substantially alleviate the symptoms of GERD or LPRD and to produce at least partial healing of esophageal erosions, if present. Preferably, treatment of the combination of a PPI and an H₂RA according to the invention is given for at least one week (or two weeks) according to an embodiment of the invention. After combination treatment has been completed, maintenance or continued therapy may continue using PPI monotherapy for 1, 2, 3, 4, 5, 6, 7, 8 weeks or longer, for example 12 weeks or indefinitely as a maintenance treatment. The term “indefinitely,” with respect to a length of treatment as used herein refers to a continuation of treatment for months or years without a predetermined stopping point, as a maintenance therapy, which may continue for life or until the treatment physician deems it no longer necessary. This PPI monotherapy may be administered transdermally or orally and preferably continues for about 5 to about 25 days, about 7 to about 21 days or about 1 to about 3 weeks after administration of the combination therapy.

Therefore, in summary, the preferred methods according to the invention include treatments involving a combination treatment of a PPI and an H₂RA (in oral, transdermal or both formulations) for a period of about 5-15 days, followed by continued administration of a PPI for an additional period of time, for example about 7-84 days or about 1-12 weeks. The total treatment regimen therefore preferably has a duration of about 14 days to a month, but may continue longer. Preferably, the treatment consists of a short course of combination therapy, lasting 1 week, followed by 1-11 weeks of PPI monotherapy. The most preferred treatment regimens according to the invention involve a one-week course of combination therapy followed by a one- or three-week course of PPI monotherapy. If necessary, however, PPI monotherapy may continue indefinitely.

The invention described above is further exemplified by the non-limiting examples below.

### EXAMPLES

### Example 1

### Exemplary and Preferred Drug Compositions

Preferred transdermal and oral drug compositions according to the invention contain a proton pump inhibitor drug selected from omeprazole, esomeprazole, lansoprazole, raveprazole and pantoprazole in a dose to provide a steady-state plasma concentration of drug of about 1.0 μmol/L to about 10.0 μmol/L (or about 2.0 μmol/L to about 8.0 μmol/L; or about 3.0 μmol/L to about 5.0 μmol/L) and a histamine H₂ receptor antagonist selected from ranitidine, famotidine, nizatidine and cimetidine in a dose to provide a steady-state plasma concentration of drug of about 30 ng/mL to about 750 ng/mL (or about 50 ng/mL to about 500 ng/mL; or about 100 ng/mL to about 400 ng/mL) in a single dosage form or in two separate dosage forms. One or both of the dosage forms may be for transdermal administration. Preferred drug compositions contain esomeprazole or lansoprazole and famotidine or ranitidine in a single oral or transdermal drug delivery device.

TABLE I
Exemplary Transdermal and Oral Drug Delivery Systems.

| Drug Device(s) | PPI | H₂RA |
|---|---|---|
| A (single) | omeprazole | ranitidine |
| B (single) | lansoprazole | famotidine |
| C (single) | raveprazole | nizatidine |
| D (dual) | pantoprazole | cimetidine |
| E (dual) | esomeprazole | cimetidine |
| F (dual) | esomeprazole | ranitidine |

### Example 2

### Exemplary and Preferred Kits for Initial Treatment of Gastroesophageal Reflux Disease and Laryngopharyngeal Reflux

Preferred kits according to the invention contain a supply of both a proton pump inhibitor drug and a histamine H₂ receptor antagonist drug sufficient for one week, for two weeks, for four weeks or sufficient to produce symptom relief and/or substantial healing of esophageal erosion and also optionally contain an additional supply of a proton pump inhibitor drug sufficient for maintained (mono)therapy for one week, for two weeks, for three weeks or longer.

In some embodiments, the kits supply a proton pump inhibitor drug selected from omeprazole, esomeprazole, lansoprazole, raveprazole and pantoprazole, in a transdermal drug delivery device or an oral dosage form at a dose to provide a steady-state plasma concentration of drug of 1.0 μmol/L to about 10.0 μmol/L (or about 2.0 μmol/L to about 8.0 μmol/L; or about 3.0 μmol/L to about 5.0 μmol/L) and a histamine H₂ receptor antagonist in a transdermal drug delivery device or oral dosage form selected from ranitidine, famotidine, nizatidine and cimetidine in a dose to provide a steady-state plasma concentration of drug of 30 ng/mL to about 750 ng/mL (or about 50 ng/mL to about 500 ng/mL; or about 100 ng/mL to about 400 ng/mL). The drugs are supplied in the form of (1) one or more transdermal drug delivery devices containing both a proton pump inhibitor and a histamine H₂ receptor antagonist, (2) at least two different transdermal drug delivery devices, one which administers a proton pump inhibitor and one which administers a histamine H₂ receptor antagonist, (3) a supply of histamine H₂ receptor antagonist formulated for oral administration (for example, tablets) and one or more transdermal drug delivery devices containing a proton pump inhibitor drug, (4) a supply of a proton pump inhibitor drug formulated for oral administration and one or more transdermal drug delivery devices containing a histamine H₂ receptor antagonist or (5) supplies of both a proton pump inhibitor drug and a histamine H₂ receptor antagonist formulated for oral administration in separate dosage forms, as a fixed combination or both.

Preferably, the kit contains drugs supplied in the form of one or more transdermal drug delivery devices containing both a proton pump inhibitor and a histamine H₂ receptor antagonist or a supply of tablets or other oral dosage form containing both a proton pump inhibitor drug and a histamine H₂ receptor antagonist. Preferred kits contain esomeprazole or lansoprazole and famotidine or ranitidine and are provided in a fixed combination for administration of the combination therapy with an additional supply of esomeprazole or lansoprazole for continuation of treatment with a PPI alone.

TABLE II
Exemplary Kits.

| Kit | PPI | H₂RA |
|---|---|---|
| A | esomeprazole (oral) | ranitidine (oral) |
| B | omeprazole (oral) | nizatidine (transdermal) |
| C | lansoprazole (transdermal) | famotidine (transdermal) |
| D | esomeprazole (transdermal) | cimetidine (oral) |
| E | pantoprazole (oral) | ranitidine (transdermal) |
| F | esomeprazole (transdermal) | nizatadine (oral) |
| G | omeprazole (oral) | famotidine (oral) |
| H | lansoprazole (transdermal) | ranitidine (transdermal) |
| I | raveprazole (oral) | famotidine (oral) |
| J | raveprazole (transdermal) | famotidine (transdermal) |
| K | esomeprazole (oral) | cimetidine (transdermal) |

### Example 3

### Exemplary Methods of Treatment for GERD or LPRD

A patient with an initial diagnosis of non-erosive or erosive GERD or LPRD is administered a transdermal drug delivery device or an oral dosage form designed to deliver a steady-state plasma concentration of about 1.0 μmol/L to about 10.0 μmol/L esomeprazole and about 30 ng/mL to about 750 ng/mL ranitidine for a period of one or two weeks. Optionally, the patient is administered esomeprazole in the same dose for an additional period of one to three weeks after the one- or two-week combination therapy.

A patient with an initial diagnosis of GERD or LPRD is administered one or more transdermal drug delivery devices designed to deliver a steady-state plasma concentration of 1.0 μmol/L to about 10.0 μmol/L lansoprazole and an oral regimen of cimetidine according to the prior art for a period of 1-2 weeks. Optionally, the patient also is provided one or more additional transdermal drug delivery devices designated to deliver the same concentration of lansoprazole for an additional 1-3 weeks.

A patient suffering from GERD or LPRD is provided a kit containing 2-4 seven-day transdermal drug delivery devices designed to deliver a steady-state plasma concentration of 1.0 μmol/L to about 10.0 μmol/L esomeprazole and 14 40 mg tablets of famotidine.

A patient with an initial diagnosis of GERD or LPRD is provided a kit containing two seven-day transdermal drug delivery devices designed to deliver a steady-state plasma concentration of 1.0 μmol/L to about 10.0 μmol/L omeprazole and one seven-day transdermal drug delivery device designed to deliver a steady-state plasma concentration of 30 ng/mL to about 750 ng/mL ranitidine.

A patient suffering from severe GERD with esophageal erosion is administered transdermal drug delivery device(s) designed to deliver a steady-state plasma concentration of 1.0 μmol/L to about 10.0 μmol/L esomeprazole and 30 ng/mL to about 750 ng/mL famotidine for a period of one to four weeks, and then continued on esomeprazole monotherapy administered transdermally or orally for at least two weeks.

A patient with an initial diagnosis of mild, moderate or severe GERD or LPRD is provided a kit containing 7 oral dosage forms, each designed to orally deliver about 5 mg to about 100 mg esomeprazole and 25 mg to about 500 mg ranitidine in a fixed combination and 7, 14 or 21 oral dosage forms, each designed to orally deliver about 5 mg to about 100 mg esomeprazole.

A patient with an initial diagnosis of mild, moderate or severe GERD or LPRD is provided a kit containing 7 oral dosage forms, each designed to orally deliver about 40 mg esomeprazole and about 75 mg ranitidine in a fixed combination and 7, 14 or 21 oral dosage forms, each designed to orally deliver about 40 mg esomeprazole.

A patient with an initial diagnosis of GERD or LPRD is provided a kit containing one seven-day transdermal drug delivery device designed to deliver an effective amount of esomeprazole or lansoprazole and ranitidine or famotidine and one seven-day transdermal drug delivery device designed to deliver an effective amount of esomeprazole or lansoprazole.

A patient with an initial diagnosis or relapse of GERD or LPRD is provided a kit containing one seven-day transdermal drug delivery device designed to deliver an effective amount of esomeprazole or lansoprazole and ranitidine or famotidine and three seven-day transdermal drug delivery devices designed to deliver an effective amount of esomeprazole or lansoprazole.

A patient suffering from GERD or LPRD is provided a kit containing 14, 21, 28 or 30 one-day transdermal drug delivery devices designed to deliver an effective amount of lansoprazole and seven one-day transdermal drug delivery devices designed to deliver an effective amount of ranitidine.

A patient suffering from GERD or LPRD is provided a kit containing 7, 14, 21 or 28 daily oral dosage forms, each designed to deliver about 5 mg to about 100 mg esomeprazole and 7 daily oral dosage forms, each designed to deliver about 25 mg to about 500 mg ranitidine.

A patient suffering from chronic or initial GERD or LPRD is provided Kit A, B, C, D, E, F, G, H, I, J or K of Example 2.

1. A method for initial treatment of gastroesophageal reflux disease or laryngopharyngeal reflux disease in a mammal in need thereof, which comprises (a) administering a proton pump inhibitor drug and a histamine H₂ receptor antagonist drug concomitantly for about 5 days to about 15 days and thereafter (b) continuing to administer a proton pump inhibitor drug.

2. The method of claim 1 wherein said concomitant administration of (a) is for about 7 days to about 14 days.

3. The method of claim 1 wherein said concomitant administration of (a) is for about 1 week.

4. The method of claim 1 wherein said concomitant administration of (a) is for about 2 weeks.

5. The method of claim 1 wherein said continuing administration of a proton pump inhibitor drug of (b) is indefinite.

6. The method of claim 1 wherein said continuing administration of a proton pump inhibitor drug of (b) is for 1-12 weeks.

7. The method of claim 1 wherein said continuing administration of a proton pump inhibitor drug of (b) is for 1-3 weeks.

8. The method of claim 1 wherein said continuing administration of (b) is for about 1 week.

9. The method of claim 1 wherein said continuing administration of (b) is for about 3 weeks.

10. The method of claim 1 wherein said proton pump inhibitor drug is formulated for oral administration.

11. The method of claim 1 wherein said proton pump inhibitor drug is formulated for transdermal administration.

12. The method of claim 1 wherein said histamine H₂ receptor antagonist drug is formulated for oral administration.

13. The method of claim 1 wherein said histamine H₂ receptor antagonist drug is formulated for transdermal administration.

14. The method of claim 1 wherein said proton pump inhibitor drug and said histamine H₂ receptor antagonist drug administered in (a) are formulated as a fixed combination.

15. The method of claim 1 wherein said proton pump inhibitor drug and said histamine H₂ receptor antagonist drug administered in (a) are formulated as separate dosage units.

16. The method of claim 1 wherein said proton pump inhibitor drug is selected from the group consisting of omeprazole, esomeprazole, lansoprazole, raveprazole and pantoprazole.

17. The method of claim 1 wherein said histamine H₂ receptor antagonist drug is selected from the group consisting of ranitidine, famotidine, nizatidine and cimetidine.

18. A kit for performing the method of claim 1, which comprises:
(a) a 1-week supply of a histamine H₂ receptor antagonist drug and
(b) a 2- to 4-week supply of a proton pump inhibitor drug.

19. A kit of claim 18 wherein said proton pump inhibitor drug is selected from the group consisting of omeprazole, esomeprazole, lansoprazole, raveprazole and pantoprazole.

20. A kit of claim 18 wherein said histamine H₂ receptor antagonist drug is selected from the group consisting of ranitidine, famotidine, nizatidine and cimetidine.

21. A kit of claim 18 wherein said histamine H₂ receptor antagonist drug and said proton pump inhibitor drug are formulated for transdermal administration.

22. A kit of claim 18 wherein said histamine H₂ receptor antagonist drug and said proton pump inhibitor drug are formulated for oral administration.
